(19) United States
(12) Patent Application Publication
(10) Pub. No.: US 2014/0308297 A1
(43) Pub. Date: Oct. 16, 2014

(54) Stem Cell Fusion Model of Carcinogenesis

(71) Applicants: David T. Harris, Tucson, AZ (US); Tom C. Tsang, Tucson, AZ (US); Xianghui He, Tianjin (CN); Brian L. Pipes, Tucson, AZ (US); Michael E. Pennington, Tucson, AZ (US); Linda C. Meade-Tollin, Tucson, AZ (US)

(72) Inventors: David T. Harris, Tucson, AZ (US); Tom C. Tsang, Tucson, AZ (US); Xianghui He, Tianjin (CN); Brian L. Pipes, Tucson, AZ (US); Michael E. Pennington, Tucson, AZ (US); Linda C. Meade-Tollin, Tucson, AZ (US)

(73) Assignee: Arizona Board of Regents on behalf of the University of Arizona, Tucson, AZ (US)

(21) Appl. No.: 14/315,039

(22) Filed: Jun. 25, 2014

Related U.S. Application Data
(62) Division of application No. 13/448,251, filed on Apr. 16, 2012.
Application No. 13/448,251 is a continuation-in-part of application No. 12/064,745, filed on Jul. 31, 2008, now Pat. No. 8,158,126.
(60) Provisional application No. 60/711,249, filed on Aug. 25, 2005.

(51) Int. Cl. C07K 16/28

(52) U.S. Cl. CPC C07K 16/2854
USPC 424/172.1; 435/325

(57) ABSTRACT

Model systems and methods for exploring mechanisms of carcinogenesis and the acquisition of metastatic ability, and to provide insights into potential therapeutic targets. The systems include and methods involve fusion of a stem cell and a genetically altered cell to evaluate carcinogenesis and metastasis and for the discovery and evaluation of new therapeutic targets to inhibit metastasis and other markers of carcinogenesis.

### CROSS REFERENCE TO RELATED APPLICATIONS

This application is a divisional of U.S. patent application Ser. No. 13/448,251, filed on Apr. 16, 2012, which is a continuation-in-part application claiming priority to U.S. patent application Ser. No. 12/064,745, filed on Jul. 31, 2008, (now U.S. Pat. No. 8,158,126), which claimed priority to International Application No. PCT/US2006/033366, filed on Aug. 25, 2006, which claimed the benefit of U.S. Provisional Application No. 60/711,249, entitled “Stem Cell Fusion Model of Carcinogenesis” filed on Aug. 25, 2005, the entire contents of all of which are incorporated by reference.

### BACKGROUND OF THE INVENTION

1. Field of the Invention

The invention relates to a cell system and method for modeling, screening drugs against, and inhibiting migration of cancer cells.

2. Description of the Related Art

Cancer has been difficult to treat because of tissue heterogeneity and gene instability. As a human disease, cancer was described as early as 1600 B.C. in ancient Egyptian writings. Hippocrates, the ancient Greek physician, recognized the difference between benign and malignant tumors and named malignant tumors “carcinos.” Cancer is currently the second-leading cause of death in developed countries.

Tremendous knowledge of cancer has been accumulated since United States President Richard Nixon declared a “war on cancer” in the 1970s. Many hypotheses of cancer development have been proposed in the last two centuries. Early hypothesis included the irritation hypothesis, embryonal hypothesis and parasitic hypothesis. Later, with the establishment of experimental oncology, chemical carcinogens were identified. Dozens of oncogenes and tumor suppressor genes were discovered through molecular analysis of human and experimental animal tumors. These studies resulted in establishment of the gene mutation hypothesis, which has been dominant over the last three decades.

Despite its intrinsic elegance, the current gene mutation hypothesis has failed to explain many important features of cancer. Indeed, the limitations of the gene mutation hypothesis have been thoroughly addressed by many researchers.

Recently, the “stem cell theory of carcinogenesis” has gained momentum with insights gained from stem cell research and the discovery of “cancer stem cells.” The stem cell theory of carcinogenesis suggests that stem cells accumulate genetic mutations and become malignant cells. However, since it is still totally dependent on the gene mutation hypothesis, the stem cell theory cannot fully address what causes the distinctive features of cancer, such as invasion and metastasis.

Mutations are rare events. Mathematical models suggest that a more frequent event is required for malignant transformation. Genomic instability was proposed as the enabling characteristic of the hallmarks of cancer. As the phenotype of genomic instability, aneuploidy has been observed in nearly all solid human cancers and is difficult to explain with gene mutation hypothesis. It has been proposed that aneuploidy accounts for cancer as an autonomous mutator, but the mechanism underlying aneuploidy remains unclear.

Hence, despite the substantial progress that has been made, the origin of cancer remains enigmatic. Because current models of carcinogenesis based on the gene mutation hypothesis have limitations in explaining many aspects of cancer, a new model of multistage carcinogenesis has been put forward by the inventors in which it is proposed that cancer development involves gene mutations and cell fusions. Specifically, cancer can result from a fusion between an “altered” pre-malignant cell and a bone marrow-derived stem cell (BMDSC). “Aneuploidy,” which is a hallmark of malignancy, is a direct consequence of this cell fusion. The “stem cell fusion” model explains the remarkable similarities between malignant cells and BMDSC. This model also explains why non-mutagens can be carcinogens, and why non-mutagenic processes, such as wound healing and chronic inflammation, can promote malignant transformation.

Cancer is a frequently inexorable adversary. Cellular mechanisms involved in cancer are numerous, interdependent, and tumor-specific. Investigation of the hallmarks and the genetic interactions associated with cancer has led to a multi-step hypothesis: that accumulation of specific cellular genetic mutations can lead to carcinogenesis malignancy and metastasis (1) by still incompletely understood mechanisms. While mutations have an established role in carcinogenesis, clearly the initiation and progression of cancer involves more than genetic alterations alone. Other critical factors include the tumor microenvironment, inflammation (2), interactions with tumor stromal cells (3), (including myofibroblasts (4)) and recruitment of mesenchymal stem cells to the tumor microenvironment (5, 6).

### SUMMARY OF THE INVENTION

In one embodiment of the invention, a method for modeling cancer cell migration is disclosed. The method preferably includes providing a bone marrow derived stem cell, providing a genetically altered cell, fusing the bone marrow derived stem cell with the genetically altered cell, thereby creating a fused cell; and measuring an indicator of migration for the fused cell. Alternatively, instead of fusing the two types of cells directly, one may obtain or culture the fused cell from a previous fusion of the bone marrow derived stem cell with the genetically altered cell.

In another embodiment of the invention, a method for screening an effect of a biological or chemical agent on tumor cell migration is described. The method includes providing a fused cell derived from a fusion of a bone marrow derived stem cell with a genetically altered cell, contacting the fused cell with a biological or chemical agent, and determining whether tumor cell migration is promoted, inhibited, or unchanged.

In yet another embodiment of the invention, a method for inhibiting tumor cell migration is described and includes comprising contacting a tumor cell with an effective amount of an antibody against ubiquitin. Preferably, this antibody is MEL-14, [e.g., MEL-14-F(ab′)₂], antibody 14372 or antibody 10C2-2.

The methods of the invention represent a new and improved carcinogenesis model for in vitro studies of tumor cell migration and in vivo studies using animals with transplanted with marker-gene modified bone marrow, for example, eGFP transgeneics. Additional features and advantages of the invention will be forthcoming from the following detailed description of certain specific embodiments when read in conjunction with the accompanying drawings.

### BRIEF DESCRIPTION OF THE DRAWINGS

FIG. 1 is a schematic illustration of malignant transformation mediated by fusion between bone marrow derived stem cells and “altered” tissue cells.

FIG. 2. Bone marrow derived mesenchymal stem cells fuse with HepG2 liver cancer cells. An experimental model of cell fusion facilitated carcinogenesis utilizes polyethylene glycol-mediated cell fusion between bone marrow-derived mesenchymal stem cells and well-differentiated HepG2 hepatocellular carcinoma cells (A). HepG2 cells are fluorescently labeled with eGFP plasmid (green; medium gray) and MSCs with DiI dye (red; dark gray). Fused cells inherit both fluorescence labels (yellow; light gray) and are sorted by flow cytometry, selecting for dual labeled cells. Fluorescent images of: B) mesenchymal stem cells labeled with DiI and C) HepG2 cells transfected with the eGFP plasmid. These two progenitor cell types were fused with PEG2000 and D) sorted with flow cytometry to select for cells containing both DiI and eGFP fluorescence. The four quadrants are indicated. Quadrant 1 includes DiI fluorescence; quadrant 2 includes cells with dual fluorescence. The P2 area represents the dual labeled cells which were sorted and collected as the fused cell population. Quadrant 3 contains cells with low fluorescence and quadrant 4 represents cells with only GFP fluorescence. E) fused cell containing two nuclei and multiple cells exhibiting mesenchymal cell morphology.

FIG. 3. Fused cells exhibit increased in vitro cellular migration and invasion compared to HepG2 tumor cells, and express EMT markers and MMP activity. A) Cell migration through 8 μm transwell chambers after 16 hours. Fused cells exhibit increased motility compared to HepG2 cells (p=0.0003) and MSC (p=0.001). B) Invasion through matrigel coated 8 μm transwell chambers after 48 hours. Fused cells exhibit increased invasion compared to HepG2 cells (p=0.047) and MSC (p=0.002). C) A western blot of whole cell lysate of EMT markers and regulatory factors, E-cadherin, vimentin, Twist and Snail. B-actin levels are detected to ensure equivalent loading of protein. D) Gelatin zymograms demonstrating that fused cells secrete increased levels of MMP-2 and MMP-9 compared to HepG2.

FIG. 4. Orthotopic injection of fused cells produces multiple undifferentiated hepatic tumors. A matrigel suspension containing 3×10⁶ cells was injected into the left hepatic lobe of seven nude mice per cell type, and the mice were observed for 10 weeks. Representative examples of gross hepatic tumors formed by A) fused cells and B) HepG2 cells are shown. C) Comparison of number of hepatic tumors/liver by gross examination of livers from four surviving mice per group. Histology of hepatic tumors stained with H&E reagent, 100× magnification: D) fused cells; E) HepG2 cells. HepG2 cells form well differentiated noninvasive tumors while fused cell tumors are poorly differentiated and invasive.

FIG. 5. Fused cells generate an increased number of metastatic lesions in the lung. Lungs were harvested from 2 mice/group. Metastases were counted in a representative H&E section from each of the 5 pulmonary lobes. A representative field of each section is shown. A) fused cells; B) HepG2 cells. The arrows indicate the presence of small metastatic lesions. C) Graphical analysis of lung metastases shows fused cells developed 20 total lesions, compared to 5 total lesions observed in HepG2 cells.

FIG. 6. Model of Cell Fusion Carcinogenesis. Carcinogenesis is a multi-step process involving genetic alterations of normal cells which result in benign hyperplasia and local tissue damage (A). B) Bone marrow-derived cells, such as stem cells, macrophages and lymphocytes, are recruited to damage tissue. C) Fusion between altered cells and stem cells forms a carcinoma cell which has inherited genetic mutations from the neoplastic cell and epigenetic changes from the stem cell. D) Epigenetic traits inherited from the stem cell produce a metastatic cell.

### DESCRIPTION OF THE PREFERRED EMBODIMENTS

As used herein, a “metastasis” means the spread of cancer from one part of the body to another. A tumor formed by cells that have spread is called a “metastatic tumor” or a “metastasis.”

The term “malignant” means cancerous, i.e., abnormal cells that divide without control and that can invade nearby tissues and spread through the bloodstream and lymphatic system to other parts of the body. Characteristics such as “aneuploidy”, “metabolic shift”, “Epithelial-mesenchymal transition (EMT)”, “plasticity” and “heterogeneity” are all part of the known hallmarks acquired by cells during carcinogenesis.

The terms “altered cell” or “genetically altered cell” are defined here as any cells with genetic or epigenetic changes sufficient to skew the normal differentiation pathway of a bone marrow derived stem cell after fusion with a stem cell. “Altered” cells include so-called “initiated” (pre-malignant) cells in the multiple-step carcinogenesis model.

The term “fusion cell” means a cell formed by the fusion of an altered cell and a stem cell.

A stem cell can be bone marrow derived, umbilical cord blood derived, tissue stem cell derived, embryonic derived and/or non-stem cells induced to a stem cell state through altering expression of genes or nuclear transplantation. Additionally these stem cells or derived stem cells themselves can be genetically altered.

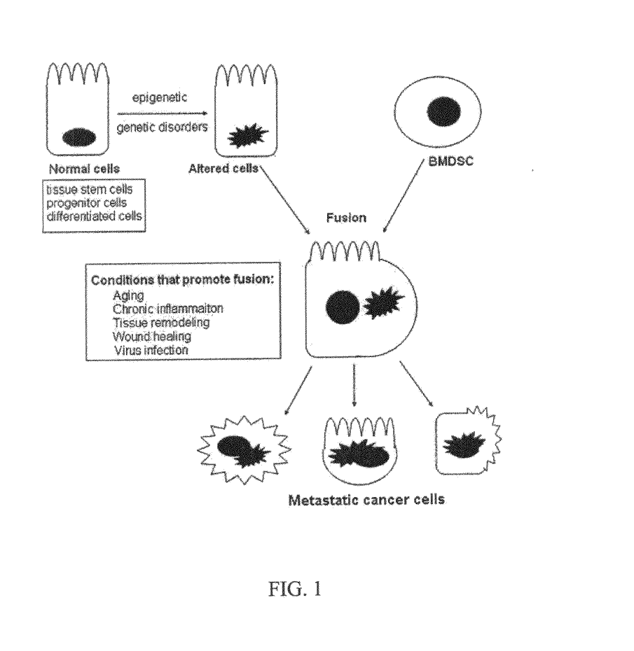

The origin of malignancy is still controversial, especially for carcinomas, which comprise more than 90% of human malignancy. The model of carcinogenesis put forward by the inventors focuses on the developmental origin of highly malignant carcinomas. The key event in this model is a fusion step between a stem cell, for example, a bone marrow-derived stem cells (BMDSC) and “altered” tissue cells (FIG. 1). Purified BMDSC populations are obtained by removing all bone marrow derived cells that expressed a differentiated cell surface markers using commercially available columns. The lineage negative cells that passes through the columns can be further enriched for stem cells by positively selecting for CD34 positive, CD133 positive and SCA-1 positive cells. This works for both mouse and human BMDSC. The invention relates in part to the recognition that the fusion between “altered” tissue cells with BMDSC may result in malignant transformation of the hybrid cells. Thus, so-called “initiated” cells in multiple step carcinogenesis model and benign tumor cells can be given the ability to migrate.

Upon fusion, the normal differentiation pathway of BMDSC could be disrupted due to the existing genetic or epigenetic disorder of the “altered” tissue cells. Genetic disorders could be gene mutations, translocations, deletions, or amplifications as proposed by the gene mutation hypothesis. Epigenetic disorders could be any change beyond the DNA sequences that result in dysregulation of cell growth and function, such as DNA methylation, chromatin modification, or altered cellular signaling. Fusion could give rise to daughter cells with the phenotype of both the altered cells and BMDSC. In other words, the daughter cells could acquire the capability of self-renewal, tissue invasion and migration from BMDSC, thereby turning into malignant cells. Moreover, the fusion process, subsequent mitosis and loss of individual copies of chromosomes, will result in aneuploidy. Aneuploidy could become the driving force of genomic instability and cancer progression. According to the inventor's model, a single fusion event could have the same transforming (from benign to malignant) effect as that of multiple events involved in the process of classical multistage carcinogenesis.

Based on the inventor's model, most of the malignant phenotype of cancer such as invasion and metastasis would come from the stem cell. Fusion is a natural and relatively frequent event during the development and maintenance of multicellular organisms. In comparing the relative probability of gene mutation and the stem cell fusion, one can view the development of a normal cell to full malignancy as an evolutionary process. Any pathway that replaces multiple rare events with a frequent event would result in such pathway to be overwhelmingly preferred in evolution.

Stem cells are highly plastic. Many studies demonstrate that bone marrow not only contains hematopoietic stem cells (HSC), but also contains mesenchymal stem cells (MSC), endothelial cell progenitors, and stem cells of epithelial tissues that can differentiate into epithelial cells of liver, lung, skin, and gastrointestinal tract. These BMDSC migrate to nonhematopoietic tissues and may play a role in maintenance and repair of damaged tissue. There are, as summarized in Table 1, striking similarities between BMDSC and metastatic cancer cells in terms of their biological activities, as well as the molecular basis of these activities.

TABLE 1
Similarity between bone marrow-derived stem cells and
metastatic cancer cells.

| Bone marrow-derived | |
|---|---|
| Stem cells | Metastatic cancer cells |
| Self-renewal | “Immortality” |
| Growth in suspension | Anchorage-independent growth |
| Multilineage differentiation | Give rise to heterogenic cancer cells |
| Migration | Invasion, metastasis |
| Extravasation | Extravasation |
| Surface markers, c-kit, | Some cancer cells express c-kit, CD34 and |
| CD34 and CD133 | CD133 |
| Chemokine receptors, such | CXCR4 expressed in metastatic tumor |
| as CXCR4 | |
| Sensitive to radiation | Poorly differentiated cancer more sensitive to |
| | radiotherapy |
| Express telomerase | Telomere maintenance |
| Partial immune privilege | Immune escape |
| Wnt and Hedgehog signal | Wnt and Hedgehog signal activity |
| activity | |
| Mediate angiogenesis | Mediate angiogenesis |

BMDSC and metastatic cancer cells are capable of self-renewal, migration, and tissue invasion. Certain cancer cells express purported stem cell markers. For example, c-kit is strongly expressed in serous ovarian carcinoma, testicular carcinoma, malignant melanoma, and small cell lung carcinoma. CD34 is expressed in dermatofibrosarcoma, epitheloid sarcoma, and solitary fibrous tumors. In addition, all types of cancer cells acquire telomere maintenance capability, similar to stem cells, which are telomerase positive. BMDSC express particular chemokine receptors and reach their destination by chemokine-ligand interactions.

Interestingly, the same chemokine-ligand pairs are involved in homing of BMDSC and malignant cell metastasis. A well-known phenomenon is that poorly differentiated cancer is usually highly metastatic but more sensitive to radiotherapy. This phenomenon has not been fully addressed in the literature, but bears remarkable resemblance to BMDSC that are highly sensitive to radiation. Indeed, this sensitivity to radiation is the basis of clinical myeloablation. Taken together, cancer cells may acquire these characteristics from, for example, a BMDSC. In fact, recent data has shown that bone marrow derived cells can give rise to gastric cancer in mice with chronic Helicobacter infections. In addition, there is a report that human skin carcinomas derived from donor cells were observed in a kidney transplant recipient.

The inventors have proposed in the past that a fusion event between, for example, a BMDSC and “altered” cells give rise to cancer cell migration. As mentioned above, fusion is a fundamental phenomenon in the life of many organisms. Intracellular vesicle fusion is essential for basic cellular function. Enveloped viruses deliver viral capsids into the cytosol through membrane fusion. From yeast to humans, life begins with fusion. Cell-cell fusion is a part of normal biological processes during the development of muscle, bone and placenta. As early as 1911, it has been proposed that malignancy could be a consequence of hybridization between leukocytes and somatic cells. Studies also showed that oncogenic transformation occurred when mammalian somatic cells took up co-cultured sperm, and/or via the experimentally-induced penetration of spermatozoa in situ. A long standing hypothesis was that hybridization of tumor cells with lymphocytes results in metastatic cells. However, prior to the invention, no one is know to have described or suggested that malignant transformation is a result of fusion between a stem cell and “altered” pre-malignant tissue cells.

Stem cells are capable of adopting the phenotype of other cells by spontaneous cell fusion. Several studies have shown that BMDSC fuse with a variety of target cells. Using a method based on Cre/lox recombination to detect cell fusion events, Alvarez-Dolado et al. (Nature 425, 968-973 [2003]) demonstrated that bone-marrow-derived cells fuse in vivo with liver hepatocytes, Purkinje neurons in the brain and cardiac muscle in the heart, resulting in the formation of multinucleated cells. Through serial transplantation of bone-marrow-derived hepatocytes, Wang et al. (Nature 422, 897-901[2003]) demonstrate that cell fusion is the principal source of bone-marrow-derived hepatocytes. Cytogenetic analysis of hepatocytes transplanted from female donor mice into male recipients demonstrated diploid to diploid fusion (80, XXXY) and diploid to tetraploid fusion (120, XXXXYY) karyotypes. In theory, fusion can occur multiple times between normal, pre-malignant and malignant cells; however, the invention specifically involves fusion between an “altered” pre-malignant tissue cell and BMDSC as a crucial step in carcinogenesis. There may be multiple fusions with the BMDSC, thereby leading to at least a tetraploid karyotype after fusion takes place.

After fusion with altered tissue cells, the normal self-renewal and differentiation of stem cells is thought to be disrupted by the abnormal signal derived from the altered cells. In contrast to other stem cell models of carcinogenesis, which propose that stem cells accumulate mutations and become transformed, the invention is consistent with the studies that show that stem cells are less tolerant to DNA damage than differentiated cells. Stem cells should be more sensitive to DNA damage in order to maintain the multipotent differentiation potential. There is no doubt that BMDSC are more sensitive to radiation than mature cells. This fact is the basis of clinical myeloablation. There is also the observation that tissue stem cells are more sensitive to killing by DNA-damaging agents. Apoptosis levels of intestinal crypt stem cells are markedly elevated by exposure to radiation or cytotoxic agents. Therefore, it is more likely that tissue cells, rather than stem cells, accumulate genetic and epigenetic disorders. After fusion with BMDSC, the daughter cells are transformed and give rise to malignant tumors.

Chromosomal abnormalities have been identified as one of the distinctive pathological features of cancer for more than 100 years. Aneuploidy has been observed in nearly all solid human cancers. In addition, clinical data suggest that the degree of aneuploidy is correlated with the severity of the diseases. An aneuploidy hypothesis of cancer emphasized the importance of aneuploidy in carcinogenesis, but the mechanism underlying aneuploidy remains unclear. In the stem cell fusion model of carcinogenesis described here, aneuploidy is an inevitable consequence of cell fusion resulting in loss of individual chromosome copies. In an earlier direct application of the proposed stem cell fusion model of carcinogenesis, studies demonstrated hyperchromasia in prostate cancer cells could be a consequence of presumptive fusion of injected spermatozoa with normal prostatic epithelial cells. Moreover, certain human precancerous lesions have shown increased frequency of tetraploid cells, such as Barrett's esophagus, ulcerative colitis, and HPV-positive atypical cervical squamous cells. Analysis of DNA ploidy demonstrates that the majority of aneuploid human prostate cancers are tetraploid. This evidence suggests that the aneuploidy of cancer originates from a tetraploidation event (i.e., fusion).

The association between chronic tissue injury, inflammation and cancer has long been observed. There are many elegant studies and reviews of the molecular and cellular mechanisms underlying this association. The inventors' interpretation of the relationship between tissue repair and carcinogenesis is as follows. Chronic tissue injury, inflammation, and subsequent tissue repair exhaust the regenerative capacity of local tissue stem cells. The local inflammatory microenvironment then favors homing of BMDSC and their involvement in tissue repair. BMDSC occasionally fuse with “altered” tissue cells and give rise to malignant transformation.

Tissues that normally undergo rapid renewal are expected to experience an increased cancer incidence, as a high turnover rate should result in local tissue stem cell exhaustion and infiltration of BMDSC. Indeed, epithelium in the skin, the lungs, and the gastrointestinal tract, which are continuously exposed to environmental insult and constantly in a state of renewal, are the tissues with a high proportion of cancers. The increased engraftment of bone marrow derived keratinocytes during wound healing has been demonstrated in sex-mismatched bone marrow transplanted mice, though the same study ruled out the presence of fusion between bone marrow-derived cells and skin epithelial cells in acute injury. Helicobacter infection is a major attributable factor in the development of gastric cancer. Chronic tissue damage and ongoing tissue repair cause an imbalance between epithelial cell proliferation and apoptosis in the stomach. Indeed, it recently was reported that bone marrow-derived cells are the origin of gastric cancer in Helicobacter-infected mice.

Aging is one of the greatest risk factors of cancer. Analysis of the age distribution of cancer resulted in the early multistage theory of carcinogenesis. Later, the gene mutation hypothesis assumed that the age distribution of cancer reflected the time required to accumulate sufficient multiple mutations for cancer development. However, an alternative explanation could be that mechanisms responsible for aging also impact stem cell function. Oxidative damage and cell senescence could enhance the frequency of improper cell-cell fusion and increase the incidence of malignancy. For instance, senescent cells compromise tissue renewal or repair, secrete factors that can alter the tissue microenvironment, and in turn could alter the activity of stem cells. In addition, stem cells themselves are also a direct target for aging-related damage. It has been demonstrated that hair graying is caused by defective self-maintenance of melanocyte stem cells. Gut epithelial stem cells have been shown to suffer important functional impairment with aging. Senescence and a functional failure of HSCs can create conditions that are permissive to leukemia development. Therefore, the chronological kinetics of carcinogenesis may reflect the cell-cell interactions during the course of aging.

Other conditions may promote cell-cell fusion and consequently increase the incidence of cancer, including tissue remodeling and virus infection. The high incidence of breast and ovarian cancer in women, and hepatocellular carcinoma following chronic hepatitis may be examples where tissue remodeling promotes malignant transformation. Epstein-Barr virus (EBV) has been shown to be associated with a wide range of cancers including Burkitt's lymphoma, non-Hodgkin's lymphoma, Hodgkin's disease, Nasopharyngeal carcinoma, gastric adenocarcinoma and breast cancer. Earlier studies have shown that EBV induces cell-cell fusion, especially by virus isolated from tumors. In concert with these data, the inventor's stem cell fusion model of carcinogenesis could explain why EBV infection associates with so many cancers.

The stem cell fusion model of carcinogenesis presented here is readily testable. Thus, several experiments that have been performed by the inventors. Fusion between benign tumor cells and BMDSC has been performed in vitro. After fusion, the morphology and capability for metastasis and invasion are determined in vitro and in vivo. Evidence of also fusion could be shown by thorough examination of the spontaneous solid tumors developed in mice receiving sex-mismatched bone marrow or transgenic bone marrow. However, because the redundant sex chromosome is often lost in the daughter cells when fusion happens, the widely used technology such as fluorescence in situ hybridization (FISH) for detecting the sex chromosome, might not be appropriate. Indeed, a considerable number of malignant tumors that develop in normal females become sex-chromatin negative, suggesting the loss of the redundant second X chromosome. Methods to detect the presence of transduced DNA species, or donor-derived mitochondria DNA might be suitable. Finally, a retrospective study could be done by examination of samples collected from previous bone marrow recipients who later developed carcinomas. Techniques, such as detection of the presence of the donor-derived mitochondrial DNA rather than FISH detecting sex chromosome, may be more informative.

The stem cell fusion model of cancer, especially carcinoma, has significant implications for cancer research and drug development, as well as for the therapeutic application of stem cells. Malignant cells might be susceptible to therapies that induce differentiation. Differentiation could switch off self-renewal activity and decrease the capability of malignant cells to metastasize and invade tissues. In fact, several differentiation-inducing agents, such as retinoic acid or peroxisome proliferators-activated receptor-gamma (PPARγ) agonists, have been used for the successful treatment of acute myeloid leukemia or liposarcoma, respectively. Introduction of a differentiation signal into malignant cells through gene transfer might be a novel viable approach for cancer therapy. In addition, metastatic cells might have a homing pattern similar to BMDSC; therefore, approaches to block BMDSC homing could be used to inhibit cancer metastasis. In agreement with this, a recent study has demonstrated that silencing of the chemokine receptor CXCR4 through RNA interference blocks breast cancer metastasis in mice. Cancer is difficult to control because its genetics are so chaotic. However, the BMDSC derived malignant characteristics of the cancer cells could present a conserved target for design of new therapies.

Thus, cancer metastasis would use the same conserved molecular mechanisms as the BMDSC and their progeny that include neutrophils, lymphocytes, and other leukocytes. Therefore, the inventors have examined whether antibodies to ubiquitin, which can block neutrophils, lymphocytes, and other leukocytes' motility and extravasation in vivo, will block cancer cell's motility and extravasation and therefore block metastasis. Furthermore, determining the presence of the of BMDSC/altered cell fusions in tumors could alert the attention of researchers to a possible unintended consequence of stem cell-based therapy (i.e., improper administration of stem cells might actually increase the incidence of malignancy).

Chronic tissue damage and subsequent repair exhaust tissue stem cells and recruit BMDSC, therefore increasing the chance for the fusion of BMDSC with tissue cells. Other factors, such as aging, viral infection and tissue remodeling, also enhance the incidence of cell fusion. Importantly, one fusion step could render multiple “malignant” characteristics to transform an “altered” cell without requiring multiple mutations.

While hundreds of studies involving fusion of tumor cells and non-tumor cells and the effect on tumorigenicity have been performed, no studies on the fusion of bone marrow-derived stem cells and tumor cells were found in the scientific literature prior to the invention.

Hence, in a first embodiment of the invention, a method for modeling cancer cell migration includes the steps of: (a) providing a bone marrow derived stem cell; (b) providing a genetically altered cell; (c) fusing the bone marrow derived stem cell with the genetically altered cell, thereby creating a fused cell; and (d) measuring an indicator of migration for the fused cell. Both BMDSC and genetically altered cells are readily available from commercial and academic tissue culture and live sources. Likewise, cell fusion is routinely practiced such that there are many protocols available (see, for example, the hybridoma protocols at protocol-online.org.). Measuring an indicator of migration for the fused cell (and it progeny) can be done through an in vitro “scratch assay” (e.g., Lal A, Glazer C A, Martinson H M, et al. Cancer Res 2002, 62:3335-3340) or through in vivo animal studies (e.g., injection of tumor cells including one or more fused cells and monitoring metastasis as described in the examples below).

The invention further involves method for screening an effect of a biological or chemical agent on tumor cell migration either in vitro or in vivo. The method includes providing a fused cell derived from a fusion of a bone marrow derived stem cell with a genetically altered cell; contacting the fused cell with a biological or chemical agent, and determining whether tumor cell migration is promoted, inhibited, or unchanged. Conserved proteins would be an especially good target for screening the effects of agents on migration.

Ubiquitin(ub) is the most conserved protein found in nature. Among its sequence of 76 amino acids, there is complete homology between species as evolutionarily divergent as insects, trout, and human. Ubiquitin makes up part of the outer surface domains of several other membrane receptors. In the case of Lymphocyte homing receptors (LHR), the presence of ub is closely correlated to LHR's function in facilitating the binding and migration of Lymphocytes through lymph nodes. All of the receptors that have been shown to be linked to ub have also been known to mediate cellular mobility. A possible explanation of these observations is that ub is involved in mediating cellular mobility through the extracellular matrix. This potential function of ub has important implications in the studies of many eukaryotic processes such as cell differentiation, parasite infection, tumor invasion and tumor cell metastasis.

Hence, for example, the biological or chemical agent is an antibody against ubiquitin, such as MEL-14 (CD62L) (available through Abcam Plc., Zymed Laboratories, et al.; see abcam.com for 21 different antibodies to ubiquitin). The cells contacted by this antibody have been subjected to a scratch assay or used in animal experiments to determine the effect of the antibody on cell migration as described below.

In another embodiment of the invention, a method for inhibiting tumor cell migration is described to include contacting a tumor cell with an effective amount of an antibody against ubiquitin. Preferably, this embodiment includes the step of confirming the presence of a fused cell among the tumor cells prior to contacting the tumor cells with the antibody so that such inhibition can be targeted to tumors with greater malignant potential.

One may determine if the tumor cell sample contains a cell with at least tetraploid DNA and at least one cell-surface marker specific to a bone marrow derived stem cell. Such surface cell markers include c-kit, CD34 and CD133 and chemokine receptors, such as CXCR4. One also may include utilizing Cre/lox recombination to detect a fusion of a bone marrow derived stem cell and a non-stem cell.

### Non-Limiting Examples

The experimental techniques to be used in these investigations are well-established and widely accepted.

The goal of this first study is to test a previously proposed hypothesis for carcinogenesis, in which the interaction of bone marrow derived stem cells and transformed cells can alter tumor progression. Two types of experiments can be performed. In the first set of experiments, cells derived from mouse bone marrow are isolated from mice which transgenically express eGFP and combined with transiently transfected transformed human or mouse cells labeled with Clontech's red fluorescent protein under conditions which facilitate the formation of hybrid cells. These hybrid cells will then be injected into a strain of mice appropriate for the cell line being tested.

Alteration of primary or metastatic tumor growth is monitored as a function of time. Two basic questions addressed by this study are whether tumor progression is modulated by the fusion of bone marrow-derived stem cells with tumor cells in various stages of transformation, and whether treatment of human or mouse xenografts with antibodies to receptors will alter the metastatic phenotypes of the xenograft tumors. A representative receptor which serves as a model to test these hypotheses is CXCR4, which is expressed by metastatic tumor cells.

Well-established xenograft models of tumor growth and progression in athymic nude, Balb/c or SCID mice must be used so that the host immune response to the administration of transformed mouse (308, 308 10Gy5, or 4T1) and human (DU145 or PC-3 M) cell lines, well-established model systems for breast, skin, and prostate cancer, will be minimal. Subcutaneous inoculation or tail vein injection is used to administer mouse cell lines into athymic nude mice. The human cell lines are administered to SCID mice. An aliquot containing cell lines, singly or in combination, are injected on day 0 and tumor growth is followed for a maximum of 40 days. Mice are then be sacrificed, tissues removed, and tumor volume and relative levels of lung metastases quantitated.

### Experiment 1.

Group A: 8 Transgenic Mice

Heterozygous transgenic eGFP mice [C57BL/6-TgN (ACTbEGFP)1Osb] (Jackson Laboratory) are used as a source of GFP labeled bone marrow cells. GFP mice are identified by expression of green fluorescence under UV light. 2- to 4-month old female heterozygotes are used as the donors for the BMT. Donor's gender is different from that of the recipient host.

Bone marrow derived cells are obtained from heterozygous GFP mice by flushing the femur and tibia with Hanks' balanced solution. To generate somatic cell hybrids, 10⁶ bone marrow-derived cells and 106 tumor cells are plated on 60 mm dishes 24 hours before treatment with polyethylene glycol (PEG). 5 grams of PEG with a molecular weight of 3000-3700 is prepared by autoclaving for 5 minutes at 121 degrees C. The autoclaved PEG is then combined with 5 ml of 2× sterile serum-free medium, pre-warmed to 37 C to prepare a 50% solution. One ml of the 50% PEG solution per dish is then added slowly to the co-cultured cells, and the cells are incubated for 1 minute at 37 degrees.

One ml of the serum-free medium is then added, and incubation continued for an additional 1 minute. Two ml of the medium is then added, and incubation continued for 2 minutes. Four ml of serum-free medium and incubation continued for 4 minutes. Medium containing serum is then added to each plate, and incubation continued for 48 hours at 37 C. After two days, each dish is passaged with trypsin and replated onto four 100 mm plates for selection. Cells expressing markers characteristic of both types of co-cultured cells are selected and grown to 90% confluence and used in subsequent experiments.

### Experiment 2: Altered Tumorigenicity and Progression of Mouse and Human Benign Tumor Cells.

Mice are inoculated with GFP-labeled bone marrow cells, singly or in combination with transformed benign human or mouse cells.

Group A: 72 mice. Strains: Athymic nude mice for 308 cells; SCID for DU145 or PC-3 M tumors (Pain category D). Total mice needed: (4 mice/treatment) (6 treatments) (3 experiments)=72 mice.

Mice are inoculated with GFP-labeled bone marrow (BM)-derived cells and/or with transformed benign human or mouse cells. Tumor inoculations are performed on mice anesthetized with isofluorane in a bell jar. The mice are placed in the jar which contains isofluorane treated cotton balls inside a polypropylene centrifuge tube. During the procedure the mice are monitored by observing respiratory rate, movement, muscle relaxation, and lack of directed movement. After inoculation, mice are returned to their cages and monitored until they regain normal consciousness.

100 ul of PBS containing 5×10⁵ cells is administered to each mouse. Athymic nude mice receive 308 cells, BM cells, or a PEG-treated mixture of BM cells and 308 cells. SCID mice receive DU145 cells, BM cells, or a PEG-treated mixture of BM cells and DU145 cells. Inoculations are administered subcutaneously or by tail vein injection. For those mice receiving tail vein infections, the mice are confined in a restraint box. After disinfection of the tail with alcohol, 2% xylacaine is applied as a topical anesthetic. No more than 200 ul of solution is injected into each mouse, using a 25-30 gauge needle. If the injections cause necrosis, the tails are sprayed with ethyl chloride, dipped in betadine, and removed with sterile scissors just above the necrotic area. The tail then is cauterized with silver nitrate to stop bleeding.

Tumor growth is monitored by caliper measurement o tumor dimensions twice weekly, and calculation of volume using the formula: Volume_{—}+½ (length)(lengtth²) Animals is sacrificed at 2, 3, and 4 weeks to monitor for the extent of metastasis and the volume of tumor achieved.

Animals are sacrificed by carbon dioxide asphyxiation in an airtight chamber in order to harvest tumors and organs. This is a routinely used procedure for euthanasia of mice that minimizes their suffering and is recommended by the AVMA Panel on Euthanasia.

### Summary Outline of Procedure

1. Administer mixture of benign transformed cells and stem cells to establish 308 and DU145 xenografts by subcutaneous or tail vein injection.

2. Treatment groups for each method of injection—(6): 308 cells; BM cells; PEG-treated mixture of BM cells+308 cells; DU145; BM cells, PEG treated DU145 and BM cell mixture.

3. Primary tumors and organs with metastases which develop will be removed after termination of the mice by CO₂ asphyxiation.

4. Submit tumor samples for histopathological analysis to detect alterations in progression or the ability to metastasize associated with a fusion event. The histopathological analysis should include comparison of tumor growth with time, relative numbers and sizes of metastases, histological characterization of the tumor.

### Experiment 3: Inhibition of Tumorigenicity or Progression.

Mice are inoculated with metastatic transformed human (PC3-M) or mouse (308 10Gy5 or 4T1) cells, and with inhibitors of the CRCX4 receptor. Total mice needed: (4 mice/treatment) (3 treatments) (3 timepoints of administration) (3 experiments)=108 mice.

Tumor inoculations are performed on mice anesthetized with isofluorane in a bell jar. The mice are placed in the jar which contains isofluorane treated cotton balls inside a polypropylene centrifuge tube. During the procedure the mice are monitored by observing respiratory rate, movement, muscle relaxation, and lack of directed movement. After inoculation, mice are returned to their cages and monitored until they regain normal consciousness.

100 ul of PBS containing 10⁴ 4T1 cells is administered injected into a mammary fat pad of 4 Balb/c mice. The athymic nude mice receive 100 ul of PBS containing 1×10⁶ 308 10Gy5 cells. The SCID mice receive 100 ul of PBS containing 1×10⁶ PC-3M cells. The experiment is performed with administration of the antibody to the CRCX4 receptor before, concurrently, and after inoculation of tumor cells. 4T1 cells are injected into Balb/c mammary fat pads. 308 10Gy5 are injected into tail veins of nude mice, and PC-3 M cells are injected into the tail veins of SCID mice. The mice receiving tail vein injections are confined in a restraint box during the injection. After disinfection of the tail with alcohol and application of 2% xylacaine as a topical anesthetic, no more than 200 ul of solution is injected into each mouse, using a 25-30 gauge needle. If the injections cause necrosis, the tails is sprayed with ethyl chloride, dipped in betadine, and removed with sterile scissors just above the necrotic area. The tail then is cauterized with silver nitrate to stop bleeding.

Tumor growth is monitored by caliper measurement of tumor dimensions twice weekly, and calculation of volume using the formula: Volume=½ (length)(length²). Animals are sacrificed at 10, 15, and 20 days to monitor for lung metastases and tumor volume.

Animals are monitored for pre- or post-inoculated with a potential inhibitor of metastasis and assayed for alterations in tumor cell apoptosis, differentiation, inhibition of metastasis.

Primary tumors and metastases which develop in the host mice are removed after termination of the mice by CO₂ asphyxiation.

Tissue samples are submitted for histopathological analysis to detect alterations in progression or metastasis associated with the treatment. The histopathological analysis should include comparison of tumor growth with time, relative numbers and sizes of metastases, histological characterization of the tumor tissue.

### In Vitro Cancer Cell/Fused Cell Migration Inhibition Assay:

Cells: Two metastatic cancer cell lines were used to test the ability of ubiquitin antibodies to inhibit cell motility. PC-3M is a human prostate carcinoma cell line. 4T1 is a mouse mammary carcinoma cell line. Both were maintained and in DMEM medium supplemented with 10% FBS and Glutamax 1 (DMEM medium).

Antibodies: Three ubiquitin antibodies were used. 14372 is a polyclonal antibody to ubiquitin. 10C2-2 and Mel-14 are both monoclonal antibodies to ubiquitin.

Procedure (1): A 6-well plate containing a sterile coverslip in each well was seeded with 1×10⁶ cells/well in DMEM medium, and incubated overnight at 37° C. and 5% CO₂, in a humidified incubator (standard conditions).

The next day, the confluent monolayer on the coverslip was scratched once with a pipette tip. The medium was aspirated and the wells were rinsed with 1 mL of DMEM medium. Each cell line was treated with three different concentrations of each antibody: 5 μg/mL/10⁶ cells, 25 μg/mL/10⁶ cells and 100 μg/mL/10⁶ cells. The plates were incubated for 11 hours with the cells. Control cells were treated with DPBS.

The coverslips were evaluated after incubation for closure of the scratches as a result of cell migration. The coverslips were then fixed and stained with 1:1 methanol:acetone for 5 minutes at −20° C. and then rinsed with DPBS. Coverslips were mounted on glass slides. Images were captured with Metacam software using a workstation composed of an Nikon TE2000 microscope at 4× magnification.

### In Vivo Cancer Cell/Fused Cell Metastasis Inhibition Assay:

Cells: A metastatic mouse mammary carcinoma cell line, 4T1, was used to test the ability of a ubiquitin antibody to inhibit metastasis. The cells were maintained in DMEM medium under the culture conditions described in the previous protocol.

### Antibodies: The Monoclonal Ubiquitin Antibody, Mel-14, was Used.

Procedure: 4T1 cells were transiently transfected with an expression vector for the enhanced green florescence protein (EGFP). Cells were harvested 48 hours after transfection and incubated with either ubiquitin antibody, Mel-14, or a control antibody, Rat IgG2A, at the concentrations of 180₁₄ per 10⁶ cells in DPBS for one hour. After incubation, 250,000 cells were injected into the tail vein of SCID mice in a total volume of 504. One week later, the mice were sacrificed and their lungs were removed and fixed in 4% formalin. Examination for the presence of metastatic colonies was performed on whole flattened lungs with a Nikon Eclipse 600 microscope at 10× magnification. The presence of EGFP positive cells in the lung indicated that metastasis has occurred.

### Results:

TABLE 2
Degree of inhibition of migration of PC3M cells in vitro by
ubiquitin antibodies at different concentrations.
(++++ = complete inhibition; − = no inhibition)

| | Conc. | | |
|---|---|---|---|
| antibodies | 5 μg/ml/10⁶ | 25 μg/ml/10⁶ | 100 μg/ml/10⁶ |
| 14372 | ++++ | ++++ | ++++ |
| 10C2-2 | ++++ | ++++ | ++++ |
| Mel-14 | ++++ | ++++ | ++++ |
| DPBS only | − | − | − |

TABLE 3
Degree of inhibition of migration of 4T1 cells in vitro by
ubiquitin antibodies at different concentrations.
(++++ = complete inhibition; − = no inhibition)

| | Conc. | | |
|---|---|---|---|
| antibodies | 5 μg/ml/million | 25 μg/ml/million | 100 μg/ml/million |
| 14372 | ++++ | ++++ | ++++ |
| 10C2-2 | ++++ | ++++ | ++++ |
| Mel-14 | ++++ | ++++ | ++++ |
| DPBS only | − | − | − |

TABLE 4
Degree of inhibition of in vivo metastasis of 4T1 cells by
ubiquitin antibody, Mel-14. (++++ = complete
inhibition; − = no inhibition)

| | Conc. |
|---|---|
| antibodies | 180 μg/10⁶ |
| Mel-14 | ++++ |
| control antibody | − |

Reference:
1. Auerbach R, Lewis R, Shinners B, Kubal L, Akhtar N. “Angiogenesis Assays: A Critical Overview” Clinical Chemistry 49 (1), 1 Jan. 2003: 32-40.

As seen in the tables above, antibodies against ubiquitin inhibited migration of tumor cells.

### Therapeutic Methods

The methods of this invention may be used to inhibit tumor migration in a subject. A vertebrate subject, preferably a mammal, more preferably a human, is administered an amount of the compound effective to inhibit tumor cell migration. The compound or pharmaceutically acceptable salt thereof is preferably administered in the form of a pharmaceutical composition.

Doses of the compounds preferably include pharmaceutical dosage units comprising an effective amount of the antibody or other agent. By an effective amount is meant an amount sufficient to achieve a steady state concentration in vivo which results in a measurable reduction in any relevant parameter of disease.

Monoclonal antibodies are now routinely used for therapy by infusion directly into the patient. The antibody can be lyophilized and stored until reconstitution with either water or saline. A dose of 4 mg/kg body weight is a typical and safe human dosage for antibody-based therapies. For example, this is an effective dose of the breast cancer antibody therapy Herceptin. Thus, in one embodiment of the invention, a human patient is dosed at 4 mg of a anti-ubiquitin antibody per kg body weight that is given intravenously.

The amount of active compound to be administered depends on the precise biological or chemical agent, the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

The foregoing compositions and treatment methods are useful for inhibiting cell migration (e.g., invasion or metastasis) in a subject having any disease or condition associated with undesired cell invasion, proliferation, metastasis.

Multiple factors are involved in carcinogenesis, but those involved in its most deadly consequence, the generation of metastases, are poorly understood. The recent awareness that interactions between motile stem cells and other cell types may contribute to the initiation of carcinogenesis and metastasis has revealed a great need for tumor-specific in vivo model systems to explore mechanisms of carcinogenesis and the acquisition of metastatic ability, and to provide insights into potential therapeutic targets. Thus, in the following disclosure, low metastatic human HepG2 cells were induced to fuse with rat bone marrow mesenchymal stem cells. Cancer-associated in vitro and in vivo properties of the fused progeny were compared with those of their progenitors.

He et al. (7) proposed a stem cell fusion model in which altered pre-malignant cells (including benign tumor cells) fuse with bone marrow-derived mesenchymal stem cells (MSCs) to form a hybrid cancer cell with properties consistent with the hallmarks of metastatic cancer (8), and with other common phenotypes associated with malignancy. The hybrid cell incorporates genetic mutations from the tumor cell and epigenetic changes from the stem cell. This model suggested that cell fusion and gene mutation are two important components of a comprehensive carcinogenesis mechanism. The stem cell fusion hypothesis proposed explanations for: 1) the remarkable similarities between malignant cells and stem cells; 2) the ability of non-mutagens to be carcinogens; 3) the ability of non-mutagenic processes, such as wound healing or chronic inflammation, to promote malignant transformation, and 4) the generation of aneuploidy and other common characteristics of malignant cancer cells.

Fused progeny cells exhibited enhanced expression of epithelial-mesenchymal transition markers (E-cadherin, vimentin) and regulatory factors (Twist, Snail), and enhanced in vitro invasion and migration. In gelatin zymography assays, matrix metalloproteinase-2 and -9 activities were elevated in fused cells compared to HepG2 cells. An in vivo xenograft assay employing orthotopic injection of fused cells into nude mice led to an increase in the number of malignant nodules in the liver and metastatic areas in the lung, compared to HepG2 cells. This model system is a flexible tool for investigation of the role of stem cell fusion in carcinogenesis and metastasis, and for the discovery and evaluation of new therapeutic targets to inhibit metastasis or that inhibit or effect other markers of carcinogenesis.

The following Abbreviations are used in the experiments described below: room temperature, rt; serum-free, s-f; mesenchymal stem cell, MSC; matrix metalloproteinase, MMP; hematoxylin and eosin, H&E.

In the present disclosure, we have performed in vitro and in vivo experiments to explore the acquisition of enhanced malignant characteristics by fused cells. These fused cells were obtained by polyethylene glycol (PEG) induced fusion of DiI-labeled rat bone marrow-derived (MSCs) with low-metastatic HepG2 human hepatoma cells (14) labeled by transfection with a plasmid expressing enhanced green fluorescent protein. The resulting dual-labeled fused cells were identified and collected by flow cytometry. The progenitor cells and their fused progeny were compared for several phenotypic characteristics of malignant cells (aneuploidy, in vitro migratory and invasive ability, EMT markers and regulatory factors, and MMP2 and MMP9 activity and in vivo formation of metastases). The experimental observations reported in this study support the proposed mechanism and role of fusion of stem cells with altered cells in the development of metastatic ability.

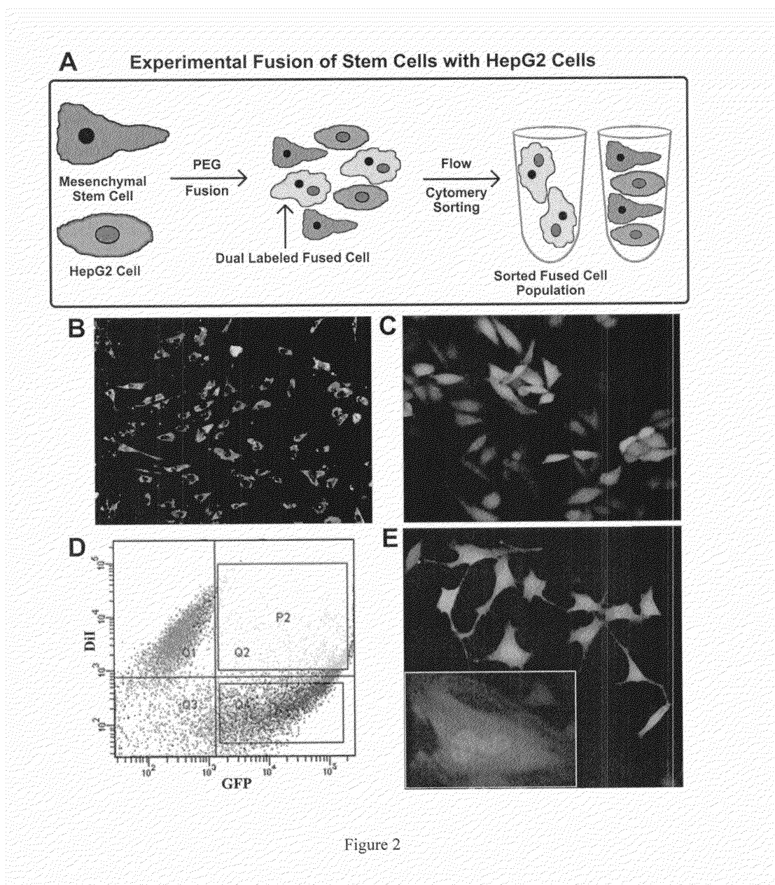

The cell fusion protocol used in this report is illustrated in (FIG. 2A). The two cell fusion progenitors are HepG2 (green), transfected with eGFP, and MSC, labeled with DiI (red). The two cell lines are combined and fused using a standard PEG fusion protocol. The small resulting population of dual-labeled fused cells is isolated from the non-fused labeled cell populations by flow cytometry.

Materials and Methods.

Fluorescent cells were monitored with an Olympus IX51 microscope (Olympus, Tokyo, Japan), fitted with an Olympus U-RFL-T fluorescent light source. Statistical analyses of the data were performed with the two-tailed Student's t test. Standard growth medium contained low glucose DMEM (1.0 g/L), (L-DMEM) (Gibco, USA), 10% FBS (Gibco, USA), 2 mM L-glutamine (Invitrogen, USA), and 100 U/ml penicillin/streptomycin. All incubations were at 37° in a 5% CO₂ humidified incubator, except where noted. In vivo experiments were carried out in accordance with EC Directive 86/609/EEC for animal experiments.

Mesenchymal Stem Cell Isolation, Culturing and Identification.

Ten 1 month-old male Sprague-Dawley rats (Experimental Animal Center, Nanjing Medical University Center) with a body mass of 80-100 g were sacrificed and sterilized in 75% ethanol for 10 min. Bone marrow was isolated under sterile conditions from femurs and tibias as previously described (15). The subsequent procedure selects for a purified population of mesenchymal stem cells. Briefly, bones were isolated and marrow was flushed out with serum-free L-DMEM and filtered through a 0.1 μm filter. Cells were propagated by incubation in standard growth medium. Non-adherent cells were removed after 4 and 24 hrs and every 2-3 days thereafter by gently washing with medium. Cultures were passed at 80-90% confluency 3 to 4 times by detachment with 0.25% trypsin for 1 minute. Flow cytometry was performed on trypsin-detached cells after washing with PBS 3 times and resuspension. Antibody reagents for cell surface markers, including CD34, CD45, CD90 and CD105 (eBiosciences, USA) were prepared according to manufacturer's instructions, and added to separate cell suspensions. The mixtures were incubated 20 m in the dark at rt. The presence of specific cell markers was identified by flow cytometry analysis.

HepG2 Cell Culture and Transfection.

Human HepG2 cells (Shanghai Institutes for Biological Sciences, CAS) have a low metastatic phenotype (14). They were cultured in high glucose DMEM, 4.5 g/L, (H-DMEM Gibco), 10% FBS, 2 mM L-glutamine and 100 U/ml penicillin/streptomycin. The cells were transfected with pEGFP-N1 plasmid, which encodes a gene for green fluorescent protein (HepG2-eGFP). Transfections were performed with lipofectamine 2000 (Invitrogen) per manufacturer's directions. Transfected cells were selected 4 weeks in 500 μg/ml G418 (Invitrogen). The fluorescence, monitored every three days, was retained during the selection and subsequent experiments.

DiI Labeling of MSC Membranes.

Confluent monolayers of MSCs were labeled for 3 hrs at 37° C. with 400 μg of DiI (Beyotime, China) in 20 ml DMEM, following manufacturer's instructions. DiI is a lipophilic carbon cyanine dye characterized by specificity, high sensitivity, and strong fluorescence on binding to plasma membranes, facilitating the observation of whole cells. The labeled cells were washed in PBS and used in the cell fusion protocol.

Cell Fusion and Selection.

A modification of the procedure of Köhler and Milstein was used (16). Briefly, 5×10⁵ MSCs were mixed with 1×10⁵ HepG2 cells and washed 3 times by resuspension and centrifugation in 30 ml s-f medium 5 min at 1000 rpm at rt. One ml of polyethylene glycol, PEG2000 (Sigma) was slowly added to the washed cell pellet. Thirty ml of s-f DMEM was added to the cell-PEG2000 mixture after 1 min. The suspension was incubated in a 37° C. water bath for 10 m to allow fusion. The cells were pelleted and resuspended in serum-containing DMEM, plated in standard growth medium, cultured 3 days, and analyzed by flow cytometry to identify and collect fused cells containing both GFP and DiI. The percentage of dual-labeled cells detected in the cell population, or efficiency of fusion, was 6.9%. Fused cells in culture retained morphology for 1-2 months.

Chromosome Analysis.

Log phase HepG2 and MSC cells suspended in in s-f medium were treated with 0.2 μg/ml colchicine and incubated 3 hrs, then treated with 75 mM KCl for 30 min in a 37° water bath, fixed with methanol/acetic acid (3:1) mixture for 1 hr at rt and dried on pre-cooled slides. Slides were stained with Giemsa solution (Invitrogen). For each cell type, 10 mitotic figures were selected, and the number of chromosomes in each figure was counted using an inverted microscope (Olympus) with a 10× objective and a 100× oil immersion lens. The average chromosome number per cell was calculated for each cell type.

Assays of Cellular Invasion and Migration.

Assays were performed in triplicate. Standard growth medium containing 20% FBS was placed in the bottom chambers of the transwell plates. The two tailed Student's t test, unmatched, was used to calculate p values for the cell counts. Migrating or invading cells were counted at 100× magnification.

Migration assays: A suspension of 20,000 HepG2 cells, MSCs or fused cells in s-f DMEM was layered on uncoated 8 μm pore size inserts seated in transwell dishes (Millipore, USA). Cells were incubated for 16 hrs at 37° C. Medium was removed from both upper and bottom chambers. The inserts were fixed 1 hr with 4% paraformaldehyde and stained with 0.1% crystal violet at rt. Cells remaining on top of the insert were removed by scraping. Seven randomly selected fields of the invaded cells on the bottom of the insert were counted.

Invasion assays: Transwell chambers (8 μm) were coated with 45 μl of a 1:8 dilution of matrigel (Becton Dickinson) in s-f DMEM, and incubated 1 hr to gel. A suspension of 40,000 cells in s-f DMEM was layered onto the inserts as described above and incubated for 36-48 hrs. The invading cells on the bottom of the inserts were stained, and seven randomly selected microscope fields were counted.

Western Blot Assay.

The fused cells, HepG2 (included for comparison with HepG2-eGFP), HepG2-eGFP and MSCs, were harvested and lysed on ice for 10 min in RIPA lysis buffer (50 mM Tris-HCl (pH 7.5), 150 mM NaCl, 0.2 mM EDTA, 1 mM phenylmethylsulfonyl fluoride, 1% NP-40 and 0.25% deoxycholate) (Beyotime, China), Protein concentration was assayed with a BCA assay kit (Pierce, USA) according to the manufacturer's instructions. The assay was as described in (17), with the following modifications. Fifty μg of proteins from each type of cell were denatured 5 min at 95° C. in sample buffer (250 mM Tris pH 6.8, 10% SDS, 50% glycerol, 5% mercaptoethanol, 0.5 mM bromphenol blue) and separated by electrophoresis in 10% SDS-PAGE gels. Equiloading of samples was detected with a 1:1000 dilution of β-actin antibody (Cell Signaling), as per manufacturer's protocol. Proteins were transferred onto polyvinylidene difluoride membranes (Perkin-Elmer). Each membrane was blocked with 5% fat-free milk at rt for 2 h, cut, and sections were incubated at 4° C. overnight with a 1:1000 dilution of one of the following primary monoclonal antibodies: anti-Vimentin (Gene Tex), anti-E-cadherin (Cell Signaling), anti-Twistl (Cell Signaling), anti-Snail (Cell Signaling) and anti-β-actin (Cell Signaling) at 4° C. After three washes for 10 min in PBS supplemented with 0.05% Tween-20 (PBST), the membrane was incubated with a peroxidase-conjugated secondary antibody (Zhongshan Golden Bridge Biotechnology) for 2 h at rt. Enhanced chemiluminescence reagent (Pierce, USA) was used for detection as per manufacturer's instructions. Gel images were scanned into a file and processed with PowerPoint software.

Gelatin Zymography Assays.

For each of the three cell types, 2×10⁵ cells were seeded in each well of a 12-well plate and incubated 12 h at 37′C. Medium was removed and 500 μl serum-free DMEM was added to each well. After incubation for 24 h, media from each plate was harvested and pooled and separated by electrophoresis in a 10% SDS-PAGE gel containing 1 mg/mL gelatin to detect MMP-2 and MMP-9 activities. After electrophoresis, gels were equilibrated in 2.5% Triton X-100 and incubated in renaturing buffer (50 mM Tris-HCl (pH 7.5), 10 mM CaCl₂, 150 mM NaCl, 1 mM ZnCl₂, and 0.02% NaN₃) for 42 h at 37′C. The gel was stained with Coomassie R250 and destained until clear bands of MMP activity were visible against the dark blue background. Activities of MMP2 and MMP9 were identified by comparing band mobility with molecular weight standards. Images were obtained by scanning gels with Alpha Innotech gel imaging systems.

Xenograft Assay.

Six-week-old BALB/c nude mice were purchased from Shanghai Laboratory Animal Company. Mice were maintained in a pathogen-free environment, under temperature-controlled conditions. Cages, bedding, and drinking water were autoclaved and changed regularly. Food was sterilized by irradiation. The mice were maintained in a daily cycle of 12 h period of alternating light and dark. The fused cells, HepG2 and MSCs were harvested, counted and centrifuged. For each cell type, 2.4×10⁷ cells were suspended in 240 μl of matrigel (Becton Dickinson, USA), and placed on ice. Twenty-one BALB/c nude male mice were randomly divided into three groups of seven mice, one group for each cell type. Before inoculation, all mice were anesthetized by intraperitoneal injection with 1% pentobarbital sodium (10 μl/g body weight) (Sigma, Germany). The peritoneal cavity was opened and the left liver lobe was exposed. The left liver lobe of each mouse was injected with thirty μl of matrigel containing 3×10⁶ cells. Implantation was finished within 6 h. The mice received gentamicin in drinking water (80,000 U/l) up to 1 week following implantation. Body mass and survival rate were calculated each week (Data not shown). Surviving mice were sacrificed after 10 weeks and necropsied to assess metastatic tumor formation. The livers, lungs, kidneys and brains of mice in each group were isolated and fixed and stained with H&E. Sections were microscopically examined by a pathologist.

Histological Preparation of Specimens.

The liver, lung, brain and kidney were removed and fixed in 10% formalin, dehydrated and embedded in paraffin. Sections of 4 μm thickness were cut and stained with hematoxylin and eosin (HE) for microscopic observation.

Results: Fusion of HepG2-EGFP Cells with MSCs Generates Progeny Cells with Enhanced Metastatic Ability.

Since MSCs do not express a single specific marker on their surfaces, identification often involves screening for multiple surface markers such as CD34, CD45, CD90 and CD105. We selected the MSCs on the basis of their differential adherent properties in culture (15). Flow cytometry analysis of MSCs showed cell surface markers were positive for CD90 and CD105 and negative for CD34 and CD45 (Data not shown). These results indicated that MSCs were not contaminated by hematopoietic cell lineages.

Since liver cancer cells can express some of the same relevant CD markers as MSC, we labeled MSC with DiI and HepG2 cells with eGFP. Rates of spontaneous fusion can vary between 1 in 10² to 1 in 10⁶ in vitro (9), and in vivo (18). Flow cytometry analysis detected 6.9% of the total cell population possessed a dual label after fusion induced by PEG2000 treatment (FIG. 2D). The sorted fused cells were collected and cultured for 2 weeks. They remained viable until use. These fused cells retained their fluorescent properties for at least one month after fusion. Fluorescence microscopy of both types of labeled progenitor cells (FIG. 2B, 2C) and the fusion hybrid (FIG. 2E) showed homogeneous labeling. Many fused cells contained two nuclei and a marked mesenchymal morphology of long cellular extrusions, shallow cell bodies, and prominent nuclei, (FIG. 2E), suggesting an epithelial-mesenchymal transition (EMT) had occurred.

Fused Cells are Aneuploid.

If fusion occurs, the fused cells should initially contain more chromosomes per cell than either progenitor cell type. Aneuploidy and enlarged nuclei commonly observed in cancer cells are a potential consequence of cellular fusion. Average numbers of Giemsa stained chromosomes/cell were compared for each type. The average chromosome counts were: MSC, 41.3+/−4.1; HepG2, 89.6+/−16.0; and fused cells, 128.9+/−21.3. The marked increase in average chromosome count, characteristic of aneuploidy, indicates that the dual-labeled fused cells contain chromosomes obtained from both MSCs and HepG2 cells. There was no extensive loss of chromosomes as a consequence of fusion over one month, the duration of observation.

Fused Cells Exhibited Increased Migration and Invasion.

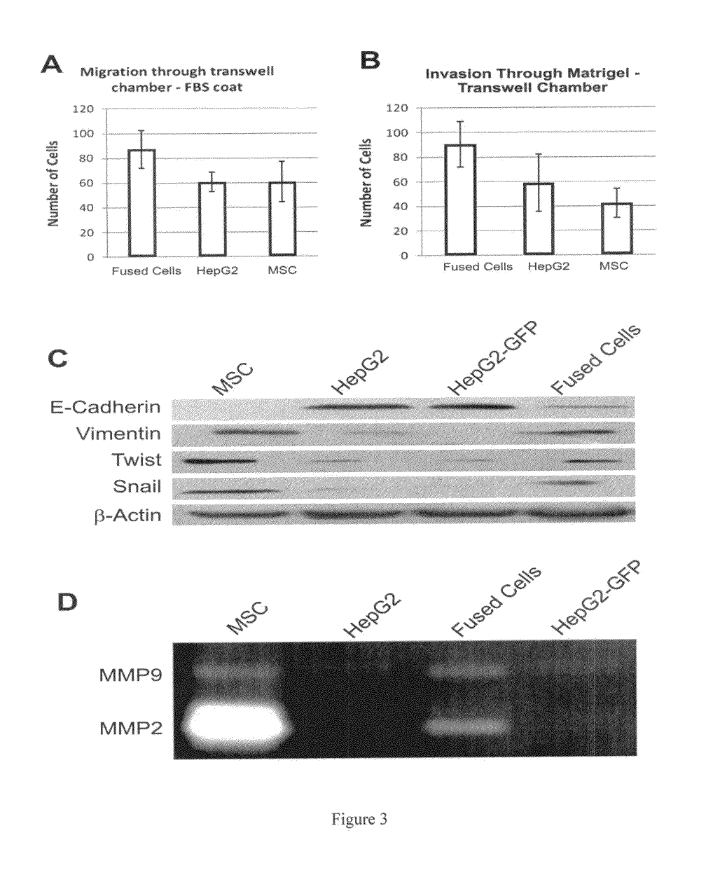

The numbers of fused cells migrating through uncoated transwell membranes were approximately 50% greater than the numbers of MSC or HepG2 cells (p=0.015431 and 0.000613, respectively) (FIG. 3A). The fused cells were also approximately 50% more invasive through matrigel-coated transwell membranes than HepG2 cells, and twice as invasive as MSC (p=0.014211 and 0.007125, respectively) (FIG. 3B).

Fused Cells Exhibit Increased EMT Marker Expression and Increased Levels of Active MMP2 and MMP9.

The fused cells exhibited morphology typical of mesenchymal cells, unlike their progenitors (FIG. 2E). Epithelial cells undergoing EMT typically lose expression of epithelial markers such as E-cadherin and increase expression of mesenchymal markers including vimentin. We wished to determine whether the fused cells might be undergoing EMT. Western blot assays detected reduced expression of E-cadherin and increased expression of Vimentin were present in fused cells compared to either HepG2 or MSCs. The EMT regulatory factors Twist and Snail were also highly expressed in fused cells and BMDSC cells, compared to HepG2/HepG2-eGFP cells, as shown in FIG. 3C. Gelatin zymography detected significantly higher activities of secreted MMP2 and MMP9 in fused cells compared with the HepG2 cells (FIG. 3D).

Fused Cells Enhance Liver and Lung Metastasis In Vivo.

The fused cells, HepG2-eGFP and MSCs were orthotopically injected into the livers of three separate groups of nude mice. After 10 weeks, the survival rate was 28.58% in the fusion group, and 57.14% in the HepG2-eGFP group.

Gross Examination.

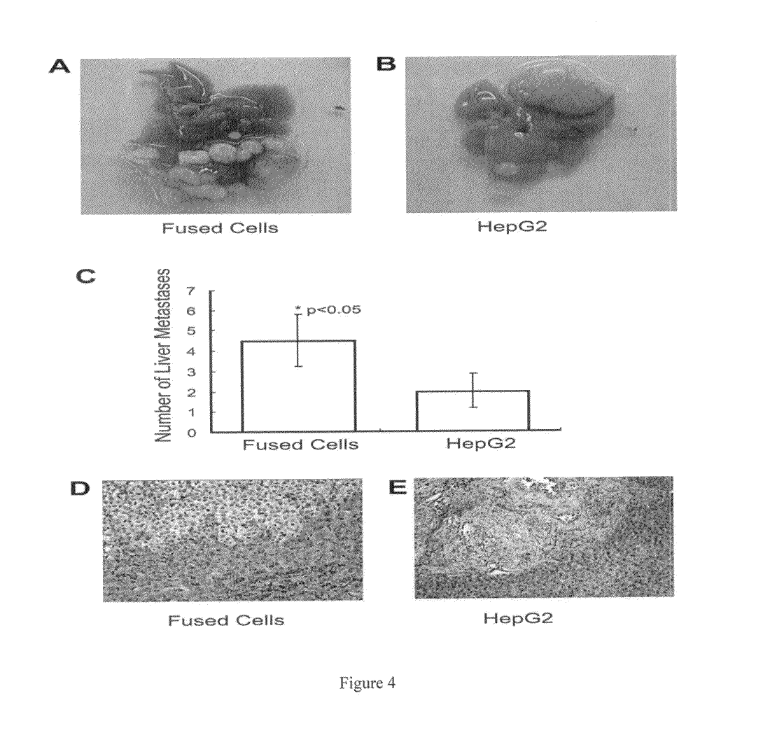

One hepatocellular carcinoma lesion at the site of orthotopic injection occurred in the left liver lobe of each mouse of the fusion group and HepG2-eGFP group. A few malignant hepatocellular carcinoma lesions were observed around the orthotopic lesion in the same liver lobe (FIGS. 4A and 4B). The average number of liver malignant lesions was 4.50±1.29 in fusion group, and was 2.0±0.82 in HepG2-eGFP group. Metastatic carcinoma lesions were present in the lungs of the fusion group and HepG2 group. The number of liver metastases showed a statistically significant difference between the 2 groups (FIG. 4C) (P<0.05) However, livers and lungs were normal in mice inoculated with MSCs.

Histological Examination.

Microscopic examination showed a hepatocellular carcinoma in a section from the fusion group, invading normal liver tissue. At high magnification, a few residual hepatocytes were present in hepatocellular carcinoma tissue (FIG. 4D). However, a well-defined hepatocellular carcinoma was observed in tissue sections from the HepG2 group. Fibrous connective tissue surrounded the hepatocellular carcinoma, so the hepatocellular carcinoma cells did not invade into the normal liver tissue (FIG. 4E).

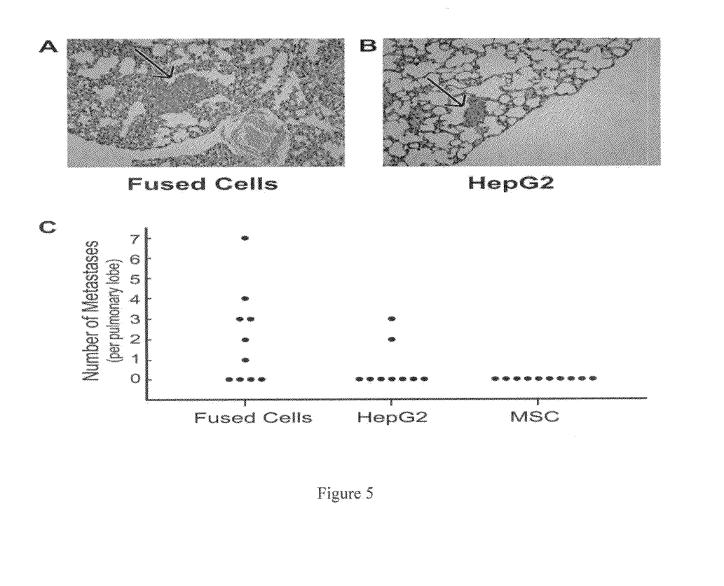

Many lung metastatic lesions were present. Representative lung tissue sections from fused cells are shown in (FIG. 5A) and HepG2 (FIG. 5B). The average number of lung metastases present was 3.29±1.89 per pulmonary lobe in tumors generated by fused cells, and was 1.25±0.75 in HepG2-eGFP inoculated mice. In FIG. 5C, the numbers of metastases detected in lung tissue from mice inoculated with parental and progeny cells are compared. The total number of lung metastatic lesions detected in lung tissue of mice inoculated with fused cells was 20. The HepG2 cells generated 5 detectable lesions. No metastases were found in kidney or brain in any mice. All organs were normal in the mice receiving MSCs. These results indicate that the fusion between HepG2-eGFP and MSCs enhanced tumor metastasis in vivo.

It has been argued that the introduction of wild type tumor suppressors into a recipient cell by fusion should reverse a loss of heterozygosity in the recipient cell which led to a more malignant phenotype, and should thus make the recipient cell less transformed and less malignant. In this study, fusion between HepG2 low-metastatic cancer cells and MSCs formed progeny cells with a more aggressive malignant phenotype than the progenitor cells. The stem cell fusion hypothesis (7) predicts that the fused cells would be both more migratory and more invasive. The fused cells exhibited increases in cell motility and invasion in vitro, expression of EMT markers and regulatory factors, and enhanced invasion and metastasis in vivo. The observed increase in the in vitro migration capacity of fused cells compared to either progenitor cell type was also reported for cells resulting from a fusion of macrophage and melanoma cells (19). Fused cells were also more invasive than MSC. Additionally, activities of the matrix metalloproteinases 2 and 9 were increased in fused cells when compared to HepG2 cells, further supporting a potentially more invasive phenotype. A similar effect, that fusing human gastric epithelial cells GES-1 with human umbilical cord derived mesenchymal stem cells results in malignant transformation of the progeny cells has been observed (unpublished data, personal communication with Dr. Xianghui He).

The type of aneuploidy observed in these experiments is a predicted consequence of cell fusion, a common characteristic of solid tumors, and is considered a hallmark of malignancy. In fact, an earlier application of the stem cell fusion model by Ablin (20-22) demonstrated hyperchromasia in prostate cancer cells as a consequence of fusion of spermatozoa with normal prostatic epithelial cells. Numerous reviews and articles have presented data and theories supporting aneuploidy as a cause of cancer (for example, (23)). Although it has been often suggested that aneuploidy is the event driving the ultimate pre-cancerous to metastatic conversion, the possibility that the fusion event actually drives the aneuploidy has received less attention.

EMT is a developmental process in which epithelial cells reduce intercellular adhesion and acquire mesenchymal properties, including down-regulation of epithelial markers (e.g. E-cadherin) and up-regulation of mesenchymal markers (e.g., vimentin, Twist, Snail) (24). Conversion of epithelial gene expression profiles to gene expression profiles of the mesoderm is an important change associated with invasion and metastasis. In certain tumors, EMT can result from tumor cell-MSC induced fusion (25). The fused cells in culture appear irregular with protruding processes, a characteristic of cells undergoing EMT. Western blot assays of fused cell protein detected a decrease in expression of E-cadherin and an increase in expression of vimentin, supporting their potential for an epithelial-mesenchymal transition. In addition, both Twist and Snail were highly expressed in the fused cells.

The matrix metalloproteinases (MMPs) are a family of ECM-degrading enzymes involved in EMT and cellular migration and invasion. Several MMPs, including MMP-2 and MMP-9, are induced in HepG2 cells by Snail (26). MMP-9 transcription and cellular invasion are induced by overexpression of Snail in MDCK cells (27). In this disclosure, both increased expression of Snail and secreted MMP-2 and -9 were observed.

The lack of suitable laboratory animal models presents a major challenge to both basic research understanding of metastasis and therapeutic development (28, 29). Fusion between genetically altered cells and stem cells and introduction into a host provides an approach to develop genetically defined and diverse models of metastasis. A recent report described specific genetic alterations of normal human lung epithelial cells which transformed them into tumorigenic cells (30). However these cells were tumorigenic, but not metastatic in a mouse model. The observations made in this in vivo xenograft assay with orthotopic injection into the livers of nude mice establishes further support for an increased metastatic phenotype in fused cells. Increased local invasiveness of fused cells compared to HepG2 cells was manifested as more numerous liver tumors as well as more metastatic lung lesions. Multiple models consisting of different cell types with specific genetic alterations fused with stem cells could be explored using the methods and cellular systems described in this disclosure. Such model systems may allow for more accurate screening and testing of therapeutic targets for prevention of the most deadly consequence of carcinogenesis: acquisition of metastatic capacity.

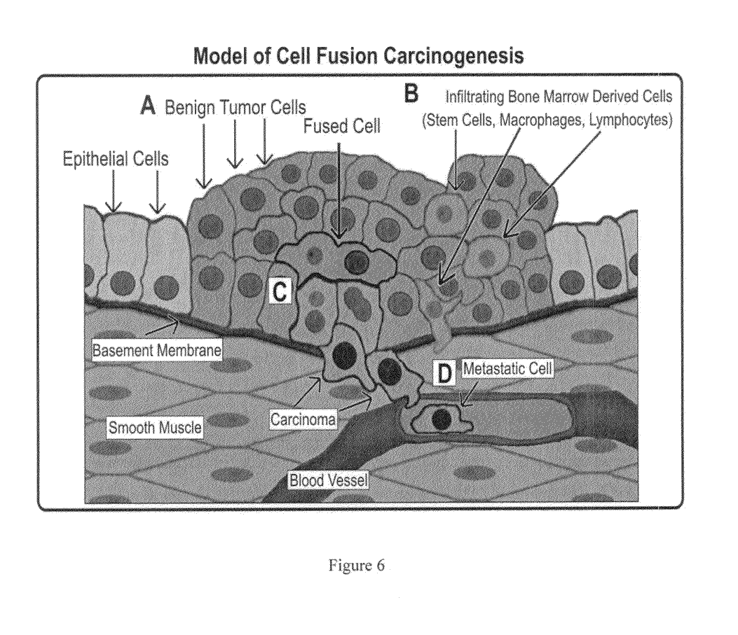

A schematic example is illustrated in FIG. 6. Carcinogenesis is a multi-step process in which genetic mutations can induce an altered hyperplastic cell phenotype which then forms a benign neoplasm and subsequent local tissue damage (FIG. 6A). Bone marrow derived cells (stem cells, macrophages and lymphocytes) are recruited to repair the damaged tissue (FIG. 6B). In the process of normal repair, fusion between an altered cell and a stem cell occurs (FIG. 6C). The resulting hybrid cell acquires epigenetic properties from the stem cell such as self-renewal, plasticity, and the capability to migrate to and survive in circulation (metastasis) while retaining both epithelial characteristics and mutations from the original tumor cell (FIG. 6D).

The fusion event could be a mechanism for transferring stem cell-like properties to the altered cell. It is important to note that traits and qualities of metastatic cells overlap significantly with those of stem cells. This is no coincidence and its importance cannot be overstated. The individual mechanistic elements imparting “stemminess” may include a variety of protein, RNA mediated and/or epigenetic controls. However, regardless of the molecular mechanism(s) responsible, the stem cell may be a wellspring from which the altered cell perverts stem cell potency into metastatic activity.

The methods described herein can be used to create new and improved carcinogenesis models. One of the uses for these models is for discovery of new therapies targeting these known cancer hallmarks.

Cell lines can be generated by fusing a genetically altered or tumor cells and stem cells, these stem cells can be bone marrow derived, umbilical cord blood derived, tissue stem cells derived, embryonic derived and/or non-stem cells induced to a stem cell state through altering expression of genes or nuclear transplantation. Additionally these stem cells or derived stem cells themselves can be genetically altered. Characteristics of cell lines derived from fusion of genetically altered/tumor cells and stem cells include epigenetic changes in gene expression such as the epithelial-mesenchymal transition, altered cell migration, aneuploidy and chromosome alterations, changes in metabolism of glucose, lactate and/or mitochondria function, and increase metastatic potential in vitro and in vivo animal models.

Fusion with stem cells and genetically altered cells provides a unique approach to develop genetically defined and diverse models of metastasis. The genetic alterations can include oncogenes, tumor suppressor genes, cell immortalizing genes such as telomerase, metabolic genes, signal transduction genes, EMT genes, drug resistant genes, cell motility genes, siRNA genes used to halt expression of any gene, and any other gene which can contribute to an altered cell phenotype.

Multiple models consisting of different cell types with specific genetic alterations fused with various different stem cells can be used to produce model systems. These models are then used for accurate screening and testing against specific therapeutic targets using various agents or treatments or combination of different agents and treatments. Most importantly these models can be used in testing of agents, treatments and combinations thereof, to treat and/or prevent the most deadly consequence of carcinogenesis: acquisition of metastatic capacity.

Various modifications are possible within the meaning and range of equivalence of the appended claims.

### REFERENCES

1. Cairns J. Cancer, Science and Society San Francisco: Freeman; 1978.
2. Coussens L M, Werb Z. Inflammation and cancer. Nat. Aust. 2002; 420:860-7.
3. Tlsty T D, Coussens L M. Tumor Stroma and Regulation of Cancer Development. Annual Review of Pathology. 2006; 1:119-50 Mech. Dis.
4. De Wever O, Demetter P, Mareel M, Bracke M. Stromal myofibroblasts are drivers of invasive cancer growth. Int J. Cancer. 2008; 123:2229-38.
5. Mishra P J, Mishra P J, Glod J W, Banerjee D. Mesenchymal Stem Cells: Flip Side of the Coin. Cancer Res. 2009; 69:1255-8.
6. Karnoub A E, Dash A B, Vo A P, Sullivan A, Brooks M W, Bell G W, et al. Mesenchymal stem cells within tumour stroma promote breast cancer metastasis. Nature. 2007; 449:557-63.
7. He X, Tsang T C, Pipes B L, Ablin R J, Harris D T. A stem cell fusion model of carcinogenesis. J Exp Ther Oncol. 2005; 5:101-9.
8. Hanahan D, Weinberg R A. The hallmarks of cancer. Cell. 2000; 100:57-70.
9. Ringertz N R, Savage R E, editors. Cell Hybrids: Academic Press; 1976.
14. Bouma M E, Rogier D, Verthier N, Lavarre C, Feldmann G. Further cellular investigation of the human hepatoblastoma-derived cell line HepG2: morphology and immunocytochemical studies of hepatic-secreted proteins. In Vitro Cell and Developmental Biology. 1989; 25:267-75.
15. Soleimani M, Nadri S. A protocol for isolation and culture of mesenchymal stem cells from mouse bone marrow. Nat. Protoc. 2009; 4:102-6.
16. Köhler G, Milstein C. Continuous cultures of fused cells secreting antibody of predefined specificity. Nature. 1975; 256:495-7.
17. Yang M-H, Chiang W-C, Chou T-Y, Chang S-Y, Chen P-m, Teng S-C, et al. Increased NBS1 Expression is a marker of aggressive head and neck cancer and overexpression of NBS1 contributes to Transformation. Clin Cancer Res. 2006; 12:507-15.
18. Duelli D, Lazebnik Y. Cell Fusion: A hidden enemy? Cancer Cell. 2003; 3:445-8.
19. Rachkovsky M, Pawelek J. Acquired melanocyte stimulating hormone-inducible chemotaxis following macrophage fusion with Cloudman S91 melanoma cells. Cell Growth Differ. 1999; 10:515-24.
20. Stein-Werblowsky R, Ablin R J. Consideraton of the possible etiology of prostatic cancer. II International Congress of Andrology; 1981; Tel Aviv, Israel; 1981.
21. Ablin R J, Stein-Werblowsky R. Toward understanding the etiology of prostate cancer. Symposium on Immunoandrology, II Pan American Congress of Andrology; 1981 Jan. 26-30; Mexico City, Mexico; 1981.
22. Ablin R J. Prostatic Cancer. In: Ablin R J, editor. Perspectives (of Prostatic Cancer). New York: Marcel Dekker; 1981.
23. Margolis R L. Tetraploidy and tumor development. Cancer Cell. 2005; 8:353-4.
24. Nieto M A. Epithelial-mesenchymal transitions in development and disease; old views and new perspectives. Int J Dev Biol. 2009; 53:1541-7.
25. Gavert N, Ben-Ze'ev A. Epithelial-mesenchymal transition and the invasive potential of tumors. Trends Mol. Med. 2008; 14:199-209.
26. Miyoshi A, Kitajima Y, Sumi K, Sato K, Hagiwara A, Koga Y, et al. Snail and SIP1 increase cancer invasion by upregulating MMP family in hepatocellular carcinoma cells. Br J. Cancer. 2005; 90:1265-73.
27. Jorda M, Olmeda D, Vinyals A, Valero E, Cubillo E, Llorens A, et al. Upregulation of MMP-9 in MDCK epithelial cell line in response to expression of the Snail transcription factor. J Cell Sci. 2005; 118:3371-85.
28. Parsons J T, Zetter B, Mohla S. Shifting Paradigms in Tumor Metastasis: Challenges and Opportunities. Cancer Biology & Therapy. 2002; 1:582-5.
29. Goldstein R H, Weinberg R A, Rosenblatt M. Of mice and (wo)men: mouse models of breast cancer metastasis to bone. J Bone Miner Res. 2010; 25:431-6.
30. Sasai K, Sukezane T, Yanagita E, Nakagawa H, Hotta A, Itoh T, et al. Oncogene-mediated human lung epithelial cell transformation produces adenocarcinoma phenotypes in vivo. Cancer Res. 2011; 71:2541-9.

We claim:

1. A composition comprising, an antibody against ubiquitin in a pharmaceutically acceptable carrier and one or more tumor cells.

2. The composition of claim 1, wherein said antibody against ubiquitin is present in an amount of between approximately 5 μg/ml/million tumor cells to 180 μg/ml/million tumor cells.

3. The composition of claim 1, wherein said tumor cells are from a carcinoma or carcinoma cell line.

4. The composition of claim 3, wherein said carcinoma cell is selected from the group consisting of breast, prostate, and skin cells.

5. A composition comprising about 4 mg of an antibody against ubiquitin in a pharmaceutically acceptable carrier per kilogram of body weight.

6. In a syringe, a dosage of an antibody against ubiquitin in a pharmaceutically acceptable carrier for intravenous administration, wherein said dosage comprises about 4 mg of antibody per kilogram of body weight of a mammal.
